(19) United States
(12) Patent Application Publication
(10) Pub. No.: US 2024/0337664 A1
(43) Pub. Date: Oct. 10, 2024

(54) CHARACTERIZATION OF CROSSLINKING SITES IN ANTIBODY-DRUG CONJUGATES

(71) Applicant: Regeneron Pharmaceuticals, Inc., Tarrytown, NY (US)

(72) Inventors: Haibo Qiu, Millwood, NY (US); Sunnie Kim, Old Tappan, NJ (US); Xiang Zheng, Allendale, NJ (US); Yimeng Zhao, Cresskill, NJ (US)

(21) Appl. No.: 18/617,922

(22) Filed: Mar. 27, 2024

Related U.S. Application Data
(60) Provisional application No. 63/457,829, filed on Apr. 7, 2023.

(51) Int. Cl. G01N 33/68; C12N 9/10; C12N 9/48; C12N 9/76; G01N 30/02; G01N 30/72

(52) U.S. Cl. CPC G01N 33/6857; C12N 9/1044; C12N 9/485; C12N 9/6427; G01N 30/7233; G01N 33/6842; G01N 33/6848; C12Y 304/17002; G01N 2030/027

(57) ABSTRACT

The present invention generally pertains to methods of characterizing crosslinking sites of a protein of interest. In particular, the present invention pertains to the use of size exclusion chromatography, peptide mapping and subunit analysis to identify and quantify crosslinking sites of a protein of interest and determine a contribution of crosslinking to the formation of high molecular weight species.

### CROSS-REFERENCE TO RELATED APPLICATIONS

This application claims the benefit of U.S. Provisional Application No. 63/457,829, filed Apr. 7, 2023, which is incorporated by reference herein in its entirety.

### BACKGROUND

Antibody-drug conjugates (ADCs) are an important class of biotherapeutics used for cancer treatment, and could also be used for therapeutic applications beyond oncology. Antibodies are covalently conjugated to payloads through either non-specific or site-specific conjugations. Conjugations through lysine (K) or reduced cysteine residues are the most common non-specific conjugation methods, which may result in a highly heterogeneous mixture of ADCs with varying drug-to-antibody ratios (DARs) and sites of payload conjugation. Given the complex nature of the product as well as problems associated with lot-to-lot variability, non-specific conjugation often leads to uncertainties in pharmacokinetics, toxicology, and efficacy. By contrast, although it usually requires more sophisticated protein engineering, site-specific conjugation generally provides superior control of the DAR and site of conjugation. While conjugation through engineered cysteine or unnatural amino acid residues are two major classes of site-specific conjugation, conjugation through enzymatic linkages are an emerging tool used to ensure control of the DAR and conjugation sites.

Microbial transglutaminase (mTG) is an enzyme that catalyzes the formation of stable isopeptide bonds between glutamine (Q) side chains (i.e., γ-carboxamide groups) and primary amines. While most published studies focus on mAb engineering to achieve higher site specificity and selectivity, it has not been extensively studied how side reactions that may occur during mTG-mediated conjugation affect the final ADC product quality. The target glutamine residue in a mAb incubated with mTG and its linker payload (LP) can undergo one of three potential reactions: (1) it can conjugate with the linker payload, (2) it can undergo deamidation by reacting with water, or (3) it can become crosslinked to a lysine (K) residue. The latter two reactions are both undesired side reactions that will reduce the number of glutamine residues available for conjugation and lead to a lower overall DAR.

Crosslinking to an off-target amino acid residue also increases the level of high molecular weight (HMW) species in the ADC products. HMW species are a critical quality attribute of therapeutic proteins due to their potential impact on both drug efficacy and safety. With size ranges from soluble oligomers to visible particles, HMW species could potentially elicit unwanted immunogenic responses, compromising a drug's safety and efficacy.

Therefore, a demand exists for methods for characterizing high molecular weight species and crosslinking side products in ADCs, for producing ADCs with reduced HMW species, and for selecting antibodies for ADCs that are less susceptible to off-target crosslinking and HMW formation.

### SUMMARY

A method has been developed for producing ADCs with reduced HMW species. In an exemplary embodiment, at least one off-target amino acid residue that forms crosslinks in the ADC can be identified by contacting the corresponding antibody to a linker and a crosslinking agent, digesting the crosslinked sample to form a peptide digest, and using liquid chromatography-mass spectrometry (LC-MS) analysis. The antibody may then be contacted to a protease to remove the off-target amino acid residue. In one aspect, the protease may be a carboxypeptidase, for example carboxypeptidase B, and the off-target amino acid residue may be a C-terminal lysine. The clipped antibody may then be contacted to a crosslinking agent and a linker-payload to produce an antibody-drug conjugate with reduced HMW species. In one aspect, the crosslinking agent may be microbial transglutaminase.

A method has also been developed for determining a contribution of site-specific crosslinking to HMW species of a protein of interest, for example an ADC. In an exemplary embodiment, the protein of interest can be subjected to conditions suitable for promoting site-specific crosslinking to produce a crosslinked protein of interest. In one aspect, the conditions suitable for promoting site-specific crosslinking can include contacting the protein of interest to a crosslinking agent, for example microbial transglutaminase. The crosslinked protein of interest can be subjected to analysis to quantify a percentage of HMW species, for example SEC analysis. In parallel or in sequence, the crosslinked protein of interest can be subjected to analysis to quantify a percentage of site-specific crosslinked peptides, for example peptide mapping analysis or subunit analysis. The percentage of site-specific crosslinked peptides can be compared to the percentage of HMW species to determine the contribution of site-specific crosslinking to HMW species. In one aspect, the comparison uses a correlation equation that determines the percentage of site-specific crosslinked peptides that would be predicted if the HMW species were entirely explained by site-specific crosslinking.

A method has further been developed for selecting and/or engineering an antibody for an antibody drug conjugate. In an exemplary embodiment, a first antibody can be subjected to site-specific crosslinking to produce a crosslinked antibody. The crosslinked antibody can be subjected to analysis to quantify site-specific crosslinking, for example peptide mapping analysis or subunit analysis. This quantification can be compared to the same quantification for at least one additional antibody, and the comparison can be used to select an antibody for an antibody-drug conjugate. In one aspect, an antibody can be selected based on forming less site-specific crosslinking. In another aspect, an antibody can be selected based on having fewer reactive off-target amino acid residues, for example reactive lysines. Additionally, or alternatively, an antibody may be engineered to remove reactive off-target amino acid residues identified using this method, thereby reducing the number of site-specific crosslinks formed by the engineered antibody.

This disclosure provides methods for producing an antibody-drug conjugate with reduced high molecular weight (HMW) species. In some exemplary embodiments, the methods can comprise: (a) contacting an antibody including a C-terminal lysine to a carboxypeptidase to produce a clipped antibody, wherein said clipped antibody does not include said C-terminal lysine; and (b) contacting said clipped antibody to a crosslinking agent and a linker-payload to produce an antibody-drug conjugate with reduced HMW species.

In one aspect, the carboxypeptidase is a metallo-carboxypeptidase, serine carboxypeptidase, cysteine carboxypeptidase, carboxypeptidase A, carboxypeptidase B, carboxypeptidase C, carboxypeptidase D, or carboxypeptidase E. In another aspect, the carboxypeptidase is carboxypeptidase B.

In one aspect, the step of contacting said antibody to said carboxypeptidase is conducted for about 1 hour to about 3 hours at about 35° C. to about 39° C.

In one aspect, the payload is a cytotoxic payload or a therapeutic payload.

In one aspect, the antibody includes a glutamine engineered for site-specific conjugation. In a specific aspect, the crosslinking agent is capable of crosslinking said glutamine and said C-terminal lysine.

In one aspect, the crosslinking agent is an enzyme. In a specific aspect, the enzyme is microbial transglutaminase.

This disclosure provides additional methods for producing an antibody-drug conjugate with reduced high molecular weight (HMW) species. In some exemplary embodiments, the methods can comprise: (a) identifying at least one off-target amino acid residue that forms crosslinks in an antibody-drug conjugate, said identifying comprising: (i) contacting a sample including an antibody to a linker and a crosslinking agent to produce a crosslinked sample, wherein said crosslinking agent is capable of crosslinking said antibody at a target amino acid residue to said linker, and wherein said crosslinking agent is capable of crosslinking said antibody at a target amino acid residue to said antibody at an off-target amino acid residue; (ii) contacting said crosslinked sample to at least one digestive enzyme to produce a peptide digest; (iii) subjecting said peptide digest to liquid chromatography-mass spectrometry (LC-MS) analysis to identify crosslinked peptides; (iv) using said identification to identify at least one off-target amino acid residue that forms crosslinks; (b) contacting said antibody to at least one protease to produce a clipped antibody, wherein said clipped antibody does not include said at least one identified off-target amino acid residue; and (c) contacting said clipped antibody to a linker and said crosslinking agent to produce an antibody-drug conjugate with reduced high molecular weight species.

In one aspect, the at least one off-target amino acid residue is a lysine, optionally wherein said lysine is a C-terminal lysine. In another aspect, the target amino acid residue is a lysine, a cysteine, an unnatural amino acid, or a glutamine. In a further aspect, the target amino acid residue is engineered for site-specific conjugation.

In one aspect, the linker is attached to a payload, optionally wherein said payload is a cytotoxic payload or a therapeutic payload.

In one aspect, the crosslinking agent is an enzyme. In a specific aspect, the crosslinking agent is microbial transglutaminase (mTG).

In one aspect, the at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C), outer membrane protein T (OmpT), immunoglobulin-degrading enzyme of Streptococcus pyogenes (IdeS), thermolysin, papain, pronase, V8 protease, variants thereof, biologically active fragments thereof, homologs thereof, or combinations thereof. In a specific aspect, the at least one digestive enzyme is trypsin. In another specific aspect, the at least one digestive enzyme is IdeS or a variant thereof.

In one aspect, the liquid chromatography is selected from a group consisting of reverse phase liquid chromatography, anion exchange chromatography, cation exchange chromatography, size exclusion chromatography, affinity chromatography, hydrophobic interaction chromatography, hydrophilic interaction chromatography, and mixed-mode chromatography. In another aspect, the LC-MS analysis is RPLC-MS/MS analysis.

In one aspect, the at least one protease is a carboxypeptidase. In a specific aspect, the carboxypeptidase is carboxypeptidase B.

In one aspect, the step of contacting said antibody to said at least one protease is conducted for about 1 hour to about 3 hours at about 35° C. to about 39° C.

In one aspect, the clipped antibody is an antibody lacking a C-terminal lysine.

This disclosure also provides methods for characterizing crosslinking sites in a protein of interest. In some exemplary embodiments, the methods can comprise: (a) contacting a sample including a protein of interest to a crosslinking agent to produce a crosslinked protein of interest, wherein said protein of interest includes at least one target amino acid residue that can be crosslinked by said crosslinking agent; (b) contacting said crosslinked protein of interest to at least one digestive enzyme to produce a peptide digest; (c) subjecting said peptide digest to liquid chromatography-mass spectrometry (LC-MS) analysis to characterize peptides that include a crosslink at said at least one target amino acid residue; and (d) using said characterized peptides to characterize crosslinking sites in said protein of interest.

In one aspect, the protein of interest is an antibody, a bispecific antibody, an antibody fragment, an antibody-drug conjugate, a fusion protein, or a recombinant protein.

In one aspect, the crosslinking agent is an enzyme. In a specific aspect, the crosslinking agent is microbial transglutaminase (mTG).

In one aspect, the target amino acid residue is a lysine, a cysteine, an unnatural amino acid, or a glutamine. In another aspect, the target amino acid residue is engineered for site-specific conjugation.

In one aspect, step (a) further comprises contacting said protein of interest and said crosslinking agent to a linker, wherein said crosslinking agent is capable of crosslinking said protein of interest to said linker.

In one aspect, the linker is attached to a payload, optionally wherein said payload is a cytotoxic payload or a therapeutic payload.

In one aspect, the at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C), outer membrane protein T (OmpT), immunoglobulin-degrading enzyme of Streptococcus pyogenes (IdeS), thermolysin, papain, pronase, V8 protease, variants thereof, biologically active fragments thereof, homologs thereof, or combinations thereof. In a specific aspect, the at least one digestive enzyme is trypsin. In another specific aspect, the at least one digestive enzyme is IdeS or a variant thereof.

In one aspect, the liquid chromatography is selected from a group consisting of reverse phase liquid chromatography, anion exchange chromatography, cation exchange chromatography, size exclusion chromatography, affinity chromatography, hydrophobic interaction chromatography, hydrophilic interaction chromatography, and mixed-mode chromatography. In another aspect, the LC-MS analysis is RPLC-MS/MS analysis.

In one aspect, the crosslinking sites include a lysine. In a specific aspect, the lysine is a C-terminal lysine.

In one aspect, the step of characterizing said crosslinking sites includes identifying amino acid residues that crosslink to said target amino acid residue.

This disclosure further provides methods for identifying at least one reactive lysine in a protein of interest. In some exemplary embodiments, the methods can comprise: (a) contacting a sample including a protein of interest to a crosslinking agent to produce a crosslinked protein of interest, wherein said crosslinking agent is capable of crosslinking at least one amino acid residue in said protein of interest to at least one reactive lysine in said protein of interest; (b) contacting said crosslinked protein of interest to at least one digestive enzyme to produce a peptide digest; (c) subjecting said peptide digest to liquid chromatography-mass spectrometry (LC-MS) analysis to identify peptides that include a crosslink between said at least one amino acid residue and at least one reactive lysine; and (d) using said identified peptides to identify said at least one reactive lysine in said protein of interest.

In one aspect, the protein of interest is an antibody, a bispecific antibody, an antibody fragment, an antibody-drug conjugate, a fusion protein, or a recombinant protein.

In one aspect, the crosslinking agent is an enzyme. In a specific aspect, the crosslinking agent is microbial transglutaminase (mTG).

In one aspect, the reactive lysine is a C-terminal lysine.

In one aspect, the at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C), outer membrane protein T (OmpT), immunoglobulin-degrading enzyme of Streptococcus pyogenes (IdeS), thermolysin, papain, pronase, V8 protease, variants thereof, biologically active fragments thereof, homologs thereof, or combinations thereof. In a specific aspect, the at least one digestive enzyme is trypsin. In another specific aspect, the at least one digestive enzyme is IdeS or a variant thereof.

In one aspect, the liquid chromatography is selected from a group consisting of reverse phase liquid chromatography, anion exchange chromatography, cation exchange chromatography, size exclusion chromatography, affinity chromatography, hydrophobic interaction chromatography, hydrophilic interaction chromatography, and mixed-mode chromatography. In another aspect, the LC-MS analysis is RPLC-MS/MS analysis.

This disclosure additionally provides methods for determining a contribution of site-specific crosslinking to high molecular weight species of a protein of interest. In some exemplary embodiments, the methods can comprise: (a) subjecting a protein of interest to conditions suitable for promoting site-specific crosslinking to produce a crosslinked protein of interest; (b) subjecting said crosslinked protein of interest to size exclusion chromatography (SEC) analysis to quantify a percent of high molecular weight (HMW) species; (c) using said quantification to determine a predicted percent of site-specific crosslinked peptides that may contribute to said HMW species, using Equation 1; (d) subjecting said crosslinked protein of interest of step (a) to peptide mapping analysis to quantify a percent of site-specific crosslinked peptides; and (e) comparing said quantified percent of site-specific crosslinked peptides of step (d) to said predicted percent of site-specific crosslinked peptides of step (c) to determine a contribution of site-specific crosslinking to HMW species of said protein of interest.

In one aspect, the protein of interest is an antibody, a bispecific antibody, an antibody fragment, an antibody-drug conjugate, a fusion protein, or a recombinant protein.

In one aspect, the site-specific crosslinking comprises crosslinking of an engineered amino acid residue.

In one aspect, subjecting a protein of interest to conditions suitable for promoting site-specific crosslinking includes contacting said protein of interest to a crosslinking agent. In a specific aspect, the crosslinking agent is an enzyme. In a more specific aspect, the enzyme is microbial transglutaminase (mTG).

In one aspect, the peptide mapping analysis includes contacting said crosslinked protein of interest to at least one digestive enzyme to produce a peptide digest, and then subjecting said peptide digest to RPLC-MS/MS analysis. In a specific aspect, the at least one digestive enzyme is trypsin.

This disclosure also provides methods for determining a contribution of a C-terminal lysine to formation of high molecular weight (HMW) species in an antibody-drug conjugate of interest. In some exemplary embodiments, the methods can comprise: (a) contacting an antibody corresponding to an antibody-drug conjugate of interest to a carboxypeptidase to produce a clipped antibody, wherein said antibody includes a C-terminal lysine and said clipped antibody does not include a C-terminal lysine; (b) contacting said antibody and said clipped antibody to a crosslinking agent to produce a crosslinked antibody and a crosslinked clipped antibody, wherein said crosslinking agent is capable of crosslinking at least one amino acid residue of said antibody and of said clipped antibody to a lysine; (c) subjecting said crosslinked antibody and said crosslinked clipped antibody to size exclusion chromatography (SEC) analysis to quantify HMW species of said crosslinked antibody and said crosslinked clipped antibody; and (d) comparing said quantification of HMW species of said crosslinked antibody to said quantification of HMW species of said crosslinked clipped antibody to determine a contribution of a C-terminal lysine to formation of HMW species in said antibody-drug conjugate of interest.

In one aspect, the carboxypeptidase is a metallo-carboxypeptidase, serine carboxypeptidase, cysteine carboxypeptidase, carboxypeptidase A, carboxypeptidase B, carboxypeptidase C, carboxypeptidase D, or carboxypeptidase E. In another aspect, the carboxypeptidase is carboxypeptidase B.

In one aspect, the crosslinking agent is an enzyme. In a specific aspect, the crosslinking agent is microbial transglutaminase (mTG).

This disclosure further provides methods for selecting an antibody for an antibody-drug conjugate. In some exemplary embodiments, the methods can comprise: (a) obtaining a sample including a first antibody, wherein said first antibody comprises at least one target amino acid residue that may be crosslinked by a crosslinking agent to at least one off-target amino acid residue; (b) contacting said first antibody to said crosslinking agent to produce a crosslinked antibody; (c) contacting said crosslinked antibody to at least one digestive enzyme to produce a peptide digest; (d) subjecting said peptide digest to liquid chromatography-mass spectrometry (LC-MS) analysis to quantify peptides comprising said at least one target amino acid residue crosslinked to at least one off-target amino acid residue for a first antibody; (e) repeating steps (a)-(d) with at least one additional antibody to quantify peptides comprising at least one target amino acid residue crosslinked to at least one off-target amino acid residue for at least one additional antibody; (f) comparing the quantifications of steps (d) and (c); and (g) using said comparison to select an antibody for an antibody-drug conjugate.

In one aspect, the at least one target amino acid residue is a lysine, a cysteine, an unnatural amino acid, or a glutamine. In another aspect, the at least one target amino acid residue is engineered for site-specific conjugation.

In one aspect, the at least one off-target amino acid residue is a lysine. In a specific aspect, the lysine is a C-terminal lysine.

In one aspect, the crosslinking agent is an enzyme. In a specific aspect, the crosslinking agent is microbial transglutaminase (mTG).

In one aspect, step (b) further comprises contacting said first antibody and said crosslinking agent to a linker, wherein said crosslinking agent is capable of crosslinking said first antibody to said linker. In a specific aspect, the linker is attached to a payload. In a more specific aspect, the payload is a cytotoxic payload or a therapeutic payload.

In one aspect, the at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C), outer membrane protein T (OmpT), immunoglobulin-degrading enzyme of Streptococcus pyogenes (IdeS), thermolysin, papain, pronase, V8 protease, variants thereof, biologically active fragments thereof, homologs thereof, or combinations thereof. In a specific aspect, the at least one digestive enzyme is trypsin. In another specific aspect, the at least one digestive enzyme is IdeS or a variant thereof.

In one aspect, the liquid chromatography is selected from a group consisting of reverse phase liquid chromatography, anion exchange chromatography, cation exchange chromatography, size exclusion chromatography, affinity chromatography, hydrophobic interaction chromatography, hydrophilic interaction chromatography, and mixed-mode chromatography. In another aspect, the LC-MS analysis is RPLC-MS/MS analysis

These, and other aspects of the invention will be better appreciated and understood when considered in conjunction with the following description and accompanying drawings. The following description, while indicating various embodiments and numerous specific details thereof, is given way of illustration and not of limitation. Many substitutions, modifications, additions, or rearrangements may be made within the scope of the invention.

### BRIEF DESCRIPTION OF THE DRAWINGS

FIG. 1A illustrates high molecular weight (HMW) species formed when a mAb is incubated with microbial transglutaminase (mTG), according to an exemplary embodiment.

FIG. 1B shows a correlation of HMW species identified by size exclusion chromatography (SEC) and crosslinked peptides identified by reduced peptide mapping (RPM), according to an exemplary embodiment.

FIG. 2A shows crosslinked sites at lysine (K) residues on mAb1, according to an exemplary embodiment.

FIG. 2B shows a percentage of native, deamidated, and crosslinked peptides from mAb1 after varying durations of incubation with mTG, according to an exemplary embodiment.

FIG. 2C shows crosslinked sites on mAb2, according to an exemplary embodiment.

FIG. 2D shows a percentage of native, deamidated, and crosslinked peptides from mAb2 after varying durations of incubation with mTG, according to an exemplary embodiment.

FIG. 3 illustrates the derivation of the correction factor allowing a percent of HMW species to be correlated to a percent of crosslinked peptides, according to an exemplary embodiment.

FIG. 4A shows a correlation between the percentage of crosslinked peptides measured by RPM and the percentage of crosslinked peptides predicted based on the percentage of HMW species measured by SEC for mAb1 incubated with mTG, according to an exemplary embodiment.

FIG. 4B shows a correlation between the percentage of crosslinked peptides measured by RPM and the percentage of crosslinked peptides predicted based on the percentage of HMW species measured by SEC for mAb1 incubated with mTG and a linker payload (LP), according to an exemplary embodiment.

FIG. 5 shows a percentage of HMW variants for mAb2 with and without intact heavy chain C-terminal lysine (K) residues, according to an exemplary embodiment.

FIG. 6A shows subunit analysis of mAb2 with the heavy chain C-terminal lysine, according to an exemplary embodiment.

FIG. 6B shows subunit analysis of mAb2 with the heavy chain C-terminal lysine after incubation with mTG, according to an exemplary embodiment.

FIG. 6C shows subunit analysis of mAb2 without the heavy chain C-terminal lysine, according to an exemplary embodiment.

FIG. 6D shows subunit analysis of mAb2 without the heavy chain C-terminal lysine after incubation with mTG, according to an exemplary embodiment.

FIG. 7A shows a relative abundance of crosslinked peptides by reduced peptide mapping for mAb2 with the C-terminal lysine on the heavy chain, according to an exemplary embodiment.

FIG. 7B shows a relative abundance of crosslinked peptides by reduced peptide mapping for mAb2 without the C-terminal lysine on the heavy chain, according to an exemplary embodiment.

### DETAILED DESCRIPTION

Antibody-drug conjugates (ADCs) are an important class of biotherapeutics used for cancer treatment. These drugs are designed to exploit the specificity of monoclonal antibodies (mAbs) to deliver cytotoxic payloads directly to target antigen-expressing cancer cells (Carter, P. J., and Senter, P. D. (2008) Antibody-drug conjugates for cancer therapy, Cancer journal (Sudbury, Mass.) 14, 154-169; Perez, H. L., et al. (2014) Antibody-drug conjugates: current status and future directions, Drug Discovery Today 19, 869-881; Alley, S. C., et al. (2010) Antibody-drug conjugates: targeted drug delivery for cancer, Current opinion in chemical biology 14, 529-537). The concept of ADCs can also be expanded to address applications beyond oncology, and now includes monoclonal antibodies that are conjugated with non-cytotoxic therapeutic payloads (McPherson, M. J., and Hobson, A. D. (2020) Pushing the Envelope: Advancement of ADCs Outside of Oncology, Methods in molecular biology (Clifton, N.J.) 2078, 23-36; Yu, S., et al. (2018) Next Horizons: ADCs Beyond Oncology, In Innovations for Next-Generation Antibody-Drug Conjugates (Damelin, M., Ed.), pp 321-347, Springer International Publishing, Cham).

Regardless of the nature of the payload to be delivered, it is critical for all ADCs to maintain stable covalent linkages between the antibody and its payload (Ducry, L., and Stump, B. (2010) Antibody-Drug Conjugates: Linking Cytotoxic Payloads to Monoclonal Antibodies, Bioconjugate chemistry 21, 5-13). Strategies of covalent conjugation of payloads to antibodies can be classified into two categories: non-specific and site-specific conjugations (McCombs, J. R., and Owen, S. C. (2015) Antibody drug conjugates: design and selection of linker, payload and conjugation chemistry, AAPS J 17, 339-351). Conjugations through lysine (K) (Wisdom, G. B. (2005) Conjugation of antibodies to fluorescein or rhodamine, Methods in molecular biology (Clifton, N.J.) 295, 131-134) or reduced cysteine (Sun, M. M. C., et al. (2005) Reduction-Alkylation Strategies for the Modification of Specific Monoclonal Antibody Disulfides, Bioconjugate chemistry 16, 1282-1290) residues are the most common non-specific conjugation methods, which will result in a highly heterogeneous mixture of ADCs with varying drug-to-antibody ratios (DARs) and sites of payload conjugation (Gordon, M. R., et al. (2015) Field Guide to Challenges and Opportunities in Antibody-Drug Conjugates for Chemists, Bioconjugate chemistry 26, 2198-2215; Doronina, S. O., et al. (2006) Enhanced Activity of Monomethylauristatin F through Monoclonal Antibody Delivery: Effects of Linker Technology on Efficacy and Toxicity, Bioconjugate chemistry 17, 114-124). Given the complex nature of the product as well as problems associated with lot-to-lot variability, non-specific conjugation often leads to uncertainties in pharmacokinetics, toxicology, and efficacy (Boylan, N. J., et al. (2013) Conjugation Site Heterogeneity Causes Variable Electrostatic Properties in Fc Conjugates, Bioconjugate chemistry 24, 1008-1016; Wagh, A., et al. (2018) Challenges and new frontiers in analytical characterization of antibody-drug conjugates, mAbs 10, 222-243; Acchione, M., et al. (2012) Impact of linker and conjugation chemistry on antigen binding, Fc receptor binding and thermal stability of model antibody-drug conjugates, mAbs 4, 362-372).

By contrast, although it usually requires more sophisticated protein engineering, site-specific conjugation generally provides superior control of the DAR and site of conjugation (Behrens, C. R., and Liu, B. (2014) Methods for site-specific drug conjugation to antibodies, mAbs 6, 46-53). While conjugation through engineered cysteine (Stimmel, J. B., et al. (2000) Site-specific Conjugation on Serine→Cysteine Variant Monoclonal Antibodies*, Journal of Biological Chemistry 275, 30445-30450; Junutula, J. R., et al. (2008) Site-specific conjugation of a cytotoxic drug to an antibody improves the therapeutic index, Nature Biotechnology 26, 925-932) or unnatural amino acid residues (Young, T. S., and Schultz, P. G. (2010) Beyond the canonical 20 amino acids: expanding the genetic lexicon, The Journal of biological chemistry 285, 11039-11044; Axup, J. Y., et al. (2012) Synthesis of site-specific antibody-drug conjugates using unnatural amino acids, PNAS 109, 16101-16106) are two major classes of site-specific conjugation, conjugation through enzymatic linkages are an emerging tool used to ensure control of the DAR and conjugation sites (Jeger, S., et al. (2010) Site-specific and stoichiometric modification of antibodies by bacterial transglutaminase, Angewandte Chemie (International ed. in English) 49, 9995-9997; Madej, M. P., et al. (2012) Engineering of an anti-epidermal growth factor receptor antibody to single chain format and labeling by Sortase A-mediated protein ligation, Biotechnology and bioengineering 109, 1461-1470).

Microbial transglutaminase (mTG) is an enzyme that catalyzes the formation of stable isopeptide bonds between glutamine (Q) side chains (i.e., γ-carboxamide groups) and primary amines (Jeger 2010; Schneider, H., et al. (2020) Recent progress in transglutaminase-mediated assembly of antibody-drug conjugates, Analytical biochemistry 595, 113615). Based on the specificity of mTG, it recognizes glutamine residues within consensus sequences and primary amines that are on a ligand or within flexible regions of a protein. Thus, most mTG-mediated ADCs are generated by conjugation of specific glutamine residues in the mAbs with primary amine-containing ligands (Anami, Y., and Tsuchikama, K. (2020) Transglutaminase-Mediated Conjugations, Methods in molecular biology (Clifton, N.J.) 2078, 71-82). Examples of the target glutamine conjugation sites in human IgGs include Q295 adjacent to the non-glycosylated N297 (Dennler, P., et al. (2014) Transglutaminase-Based Chemo-Enzymatic Conjugation Approach Yields Homogeneous Antibody-Drug Conjugates, Bioconjugate chemistry 25, 569-578), both Q295 and Q297 when introducing a N295Q mutation (Lhospice, F., et al. (2015) Site-Specific Conjugation of Monomethyl Auristatin E to Anti-CD30 Antibodies Improves Their Pharmacokinetics and Therapeutic Index in Rodent Models, Molecular Pharmaceutics 12, 1863-1871), and artificially-incorporated glutamine-containing peptide tags (Q-tags) (Ebenig, A., et al. (2019) Efficient Site-Specific Antibody-Drug Conjugation by Engineering a Nature-Derived Recognition Tag for Microbial Transglutaminase, Chembiochem: a European journal of chemical biology 20, 2411-2419). In addition to their capacity to react with primary amines, glutamine side chains can also react with water and undergo deamidation if a primary amine is not available at the reaction site (Anami and Tsuchikama 2020).

While most published studies focus on mAb engineering to achieve higher site specificity and selectivity, it has not been extensively studied how side reactions that may occur during mTG-mediated conjugation affect the final ADC product quality (Farias, S. E., et al. (2014) Mass spectrometric characterization of transglutaminase based site-specific antibody-drug conjugates, Bioconjugate chemistry 25, 240-250). The target glutamine residue in a mAb incubated with mTG and its linker payload (LP) can undergo one of three potential reactions: (1) it can conjugate with the linker payload, (2) it can undergo deamidation by reacting with water, or (3) it can become crosslinked to a lysine (K) residue (Martins, I. M., et al. (2014) Transglutaminases: recent achievements and new sources, Applied microbiology and biotechnology 98, 6957-6964). The latter two reactions are both undesired side reactions that will reduce the number of glutamine residues available for conjugation and lead to a lower overall DAR. Furthermore, the formation of intermolecular glutamine-lysine (Q-K) crosslinks increases the level of high molecular weight (HMW) variants in the ADC products, which has potential implications with respect to both drug potency and safety (Rosenberg, A. S. (2006) Effects of protein aggregates: An immunologic perspective, The AAPS Journal 8, E501-E507). Given that mTG-mediated Q-K crosslinking may have a direct impact on product quality, this reaction should be closely monitored during the production of all mTG-mediated ADCs and must be minimized by designing mAb sequences with lysine residues that are less accessible and thus less likely to crosslink with target glutamine residues.

In the present disclosure, the formation of HMW variants in mTG-mediated ADCs was investigated using size exclusion chromatography (SEC) and liquid chromatography-mass spectrometry (LC-MS). It was discovered that the formation of mTG-mediated Q-K crosslinks has a direct impact on the level of high molecular weight (HMW) variants in the final ADC product. A model system, in which two mAbs with glutamine engineered for site-specific conjugation were incubated with mTG in the absence of linker payload, was used to study the relationship between formation of HMW variants and Q-K crosslinks. SEC was used to determine the level of HMW variants and reduced peptide mapping (RPM) was used to identify the crosslinked sites and quantify the crosslinked peptides. By establishing a correlation between these two assays, Q-K crosslinking between target glutamine and specific lysine residues were identified as the major contributor to HMW size variants in the ADCs studied. More importantly, since the heavy chain (HC)C-terminal K was identified as a crosslink site in both mAbs, it was demonstrated that the level of HMW variants was greatly reduced in the ADCs in which the HC C-terminal K were completely removed before mTG-mediated conjugation. These results indicate that Q-K crosslinking and other side reactions take place during mTG-mediated conjugation and will need to be monitored and controlled to ensure the high quality and consistency of the final ADC product.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-KI cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein of interest can be a recombinant protein, an in vivo product of gene therapy, a therapeutic protein, an antibody, a bispecific antibody, a multispecific antibody, antibody fragment, monoclonal antibody, antigen-binding protein, fusion protein, scFv, a multisubunit protein, an antibody-drug conjugate, a receptor, a receptor ligand, and combinations thereof.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. An amino acid consensus sequence may be defined based on a side-by-side analysis of two or more CDRs. The term “antibody,” as used herein, also includes antigen-binding fragments of full antibody molecules.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F (ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology. Examples of bsAbs include those disclosed in the following patent applications, which are hereby incorporated by reference: U.S. Ser. No. 12/823,838, filed Jun. 25, 2010; U.S. Ser. No. 13/488,628, filed Jun. 5, 2012; U.S. Ser. No. 14/031,075, filed Sep. 19, 2013; U.S. Ser. No. 14/808,171, filed Jul. 24, 2015; U.S. Ser. No. 15/713,574, filed Sep. 22, 2017; U.S. Ser. No. 15/713,569, field Sep. 22, 2017; U.S. Ser. No. 15/386,453, filed Dec. 21, 2016; U.S. Ser. No. 15/386,443, filed Dec. 21, 2016; U.S. Ser. No. 15/22,343 filed Jul. 29, 2016; and U.S. Ser. No. 15/814,095, filed Nov. 15, 2017.

As used herein, “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, phage display technologies, gene therapy, or a combination thereof.

As used herein, the term “conjugated peptide or protein” can refer to a peptide or protein attached to biologically active drug(s) by linker(s), including “antibody-drug conjugate” or “ADC”. A conjugated peptide, a conjugated protein, or an antibody-drug conjugate can comprise several molecules of a biologically active drug (or the payload) which can be covalently linked to conjugation sites, such as side chains of amino acid residues of a conjugated peptide, a conjugated protein, or an antibody (Siler Panowski et al., Site-specific antibody drug conjugates for cancer therapy, 6 mAbs 34-45 (2013)). The development of ADCs is a strategy to improve drug efficacy, since antibodies can bind to specific sites of target cells allowing for efficient delivery of the biologically active drugs to target cells. An antibody used for an ADC can be capable of binding with sufficient affinity for selective accumulation and durable retention at a target site. Most ADCs can have Kd values in the nanomolar range. The payload can have potency in the nanomolar/picomolar range and can be capable of reaching intracellular concentrations achievable following distribution of the ADC into target tissue. The linker that forms the connection between the payload and the antibody can be capable of being sufficiently stable in circulation to take advantage of the pharmacokinetic properties of the antibody moiety (e.g., long half-life) and to allow the payload to remain attached to the antibody as it distributes into tissues, yet should allow for efficient release of the biologically active drug once the ADC can be taken up into target cells. The linker can include those that are non-cleavable during cellular processing and those that are cleavable once the ADC has reached the target site. With non-cleavable linkers, the biologically active drug released within the cell includes the payload and all elements of the linker still attached to an amino acid residue of the antibody, for example a lysine, cysteine, or glutamine residue, following complete proteolytic degradation of the ADC within the lysosome. Cleavable linkers are those whose structure includes a site of cleavage between the payload and the amino acid attachment site on the antibody. Cleavage mechanisms can include hydrolysis of acid-labile bonds in acidic intracellular compartments, enzymatic cleavage of amide or ester bonds by an intracellular protease or esterase, and reductive cleavage of disulfide bonds by the reducing environment inside cells.

The general distribution profile of ADCs contains a mixture of ADCs, unconjugated antibodies, and unconjugated drug payloads. The amount of drug which can be delivered to the target cells would decrease in the presence of unconjugated antibodies, since the unconjugated antibodies compete with drug-conjugated antibodies for the target antigens. Commonly, the derived ADCs are highly heterogeneous species containing various ADC species with variable drug-to-antibody ratios (DARs) and varied conjugation sites including conjugated conjugation sites and unconjugated conjugation sites. In the case of ADCs generated using enzymatic conjugation reactions, for example mTG-mediated conjugation, ADC species may include conjugated conjugation sites, crosslinked conjugation sites, and deamidated conjugation sites. The heterogeneity of ADCs can have significant impacts on drug safety and efficacy due to the presence of undesired ADC species. Desirable ADC formulations should include well-defined DARs and a degree of homogeneity. Quantitation and characterization of site-specific drug conjugations of ADCs with variable DARs, such as site-specific quantitation of drug conjugation, are critical processes to control the quality attributes of ADC formulations, which can directly affect the efficacy of ADCs.

A conjugated peptide or protein, for example an ADC, may be engineered to have a site-specific conjugation site, for example an engineered cysteine, an engineered unnatural amino acid residue, or an engineered glutamine. A site-specific conjugation site may also be referred to as a target site, target conjugation site, or target amino acid residue. A target amino acid residue may be, for example, an amino acid residue within a consensus sequence that is recognized by a relevant enzyme, such a glutamine residue targeted by mTG. In contrast, an off-target amino acid residue may be an amino acid residue that forms undesirable cross-links with a target amino acid residue, and does not contribute to conjugation with a linker, for example a lysine residue that is cross-linked to a target glutamine residue by mTG. An off-target amino acid residue, for example a lysine residue, may be referred to as a reactive amino acid residue, for example a reactive lysine. While a protein such as an antibody may include many amino acid residues theoretically capable of forming undesirable cross-links, for example lysines, in practice only one or a few of those amino acid residues may be reactive and participate in crosslinking reactions. The disclosure herein provides methods for designing a conjugated peptide or protein, and methods for selecting a conjugated peptide or protein, for example to avoid formation of HMW species by characterizing and reducing or eliminating potential sites of cross-linking.

In some exemplary embodiments, the protein of interest can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., HEK293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK (15) cells, GHi cells, GH3 cells, L2 cells, LLC-RC 256 cells, MHiCi cells, XC cells, MDOK cells, VSW cells, and TH-I, BI cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NSO, NS1 cells or derivatives thereof).

In some exemplary embodiments, the sample including the protein of interest can be prepared prior to or following enrichment steps, separation steps, and/or analysis steps. Preparation steps can include alkylation, reduction, denaturation, digestion, and/or deglycosylation.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl) phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds. In some exemplary embodiments, non-reduced preparation may be used, for example, in order to preserve an endogenous disulfide bond between Fab arms of an antibody or antibody-derived protein. In other exemplary embodiments, partially-reduced preparation may be used, for example, in order to reduce the disulfide bond between Fab arms of an antibody or antibody-derived protein without fully reducing the protein.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein. Non-limiting examples of hydrolyzing agents that can carry out enzymatic digestion include protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C), outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

In some exemplary embodiments, IdeS or a variant thereof is used to cleave an antibody below the hinge region, producing an Fc fragment and a Fab₂ fragment. Digestion of an analyte may be advantageous because size reduction may increase the sensitivity and specificity of characterization and detection of the analyte using LC-MS. When used for this purpose, digestion that separates out an Fc fragment and keeps a Fab₂ fragment for analysis may be preferred. This is because variable regions of interest, such as the complementarity-determining region (CDR) of an antibody, are contained in the Fab₂ fragment, while the Fc fragment may be relatively uniform between antibodies and thus provide less relevant information. Alternatively, or additionally, digestion that separates out a Fab₂ fragment and keeps an Fc fragment for analysis may be preferred, because the Fc fragment contains an N-glycosylation site of interest. Analysis of digested antibody fragments (subunits) of an antibody, for example digestion with IdeS followed by chromatographic and/or mass spectrometry analysis, is referred to as subunit analysis.

IdeS digestion has a high efficiency, allowing for high recovery of an analyte. The digestion and elution process may be performed under native conditions, allowing for simple coupling to a native LC-MS system. IdeS or variants thereof are commercially available and may be marketed as, for example, FabRICATOR® or FabRICATOR Z®.

In some exemplary embodiments, a carboxypeptidase is used to remove a carboxy-terminal (C-terminal) residue of a protein of interest. A carboxypeptidase is a protease that hydrolyzes a peptide bond at the C-terminal of a protein or peptide. Carboxypeptidases include, for example, metallo-carboxypeptidases, serine carboxypeptidases, cysteine carboxypeptidases, carboxypeptidase A, carboxypeptidase B, carboxypeptidase C, carboxypeptidase D, and carboxypeptidase E. A carboxypeptidase may be useful for removing a C-terminal lysine of a protein of interest. In other embodiments, other proteases may be used to remove residues of a protein or peptide that contribute to undesirable crosslinking, for example aminopeptidases. A protein, for example an antibody, subjected to proteolysis by a protease, for example a carboxypeptidase, may be referred to as a clipped protein, for example a clipped antibody. A clipped protein may have substantially the same or improved structure, function, safety, and efficacy as the original protein.

As used herein, a “sample” can be obtained from any step of the bioprocess, such as cell culture fluid (CCF), harvested cell culture fluid (HCCF), any step in the downstream processing, drug substance (DS), or a drug product (DP) comprising the final formulated product. In some other specific exemplary embodiments, the sample can be selected from any step of the downstream process of clarification, chromatographic production, viral inactivation, or filtration. In some specific exemplary embodiments, the drug product can be selected from manufactured drug product in the clinic, shipping, storage, or handling.

In some exemplary embodiments, the sample is a biological sample. As used herein, the term “biological sample” refers to a sample taken from a living organism, for example a human or non-human mammal. A biological sample may comprise or consist of, for example, whole blood, plasma, serum, saliva, tears, semen, check tissue, organ tissue, urine, feces, skin, or hair. A sample may be taken from a patient, for example, a clinical sample. In some exemplary embodiments, a sample may be taken from a non-human animal, for example, a preclinical sample. In some exemplary embodiments, a sample may be taken from a non-human animal subjected to gene therapy in order to produce at least one protein of interest that may be included in the sample. In some embodiments, a sample is a further processed form of any of the aforementioned examples of samples.

As used herein, the term “impurity” can include any undesirable protein present in a protein sample or protein biopharmaceutical product. Impurity can include process and product-related impurities. The impurity can further be of known structure, partially characterized, or unidentified. Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

Product-related impurities (e.g., precursors, certain degradation products) can be molecular variants arising during manufacture and/or storage that do not have properties comparable to those of the desired product with respect to activity, efficacy, and safety. Such variants may need considerable effort in isolation and characterization in order to identify the type of modification(s). Product-related impurities can include truncated forms, modified forms, and aggregates. Truncated forms are formed by hydrolytic enzymes or chemicals which catalyze the cleavage of peptide bonds. Modified forms include, but are not limited to, deamidated, isomerized, mismatched S—S linked, oxidized, or altered conjugated forms (e.g., glycosylation, phosphorylation). Modified forms can also include any post-translational modification form. Aggregates include dimers and higher multiples of the desired product. (Q6B Specifications: Test Procedures and Acceptance Criteria for Biotechnological/Biological Products, ICH August 1999, U.S. Dept. of Health and Humans Services). In some exemplary embodiments, aggregates (HMW species) may be formed by off-target crosslinking of a protein, for example an antibody in an antibody-drug conjugate.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase (RP) liquid chromatography, ion-exchange (IEX) chromatography, size exclusion chromatography (SEC), affinity chromatography, hydrophobic interaction chromatography (HIC), hydrophilic interaction chromatography (HILIC), or mixed-mode chromatography (MMC).

In some exemplary embodiments, the methods of the present invention include the use of size exclusion chromatography. Size exclusion chromatography or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase. Variants of a protein of interest that have a higher molecular weight or lower molecular weight than the main species, or than the intended product, may be referred to as “size variants.”

Analytes eluting from an SEC column may be separated into fractions based on elution time. For example, analytes eluting earlier than the functional form of a protein of interest, for example the monomeric form, may be broadly categorized as high molecular weight (HMW) species. A HMW fraction may be further subdivided into, for example, a very high molecular weight (vHMW) fraction and a dimer fraction (representing the elution time of a dimer of the protein of interest). Analytes eluting later than the functional form of a protein of interest may be broadly categorized as low molecular weight (LMW) species, and may be further subdivided into a LMW fraction and a later tail fraction.

A HMW species, such as a dimer, may be formed as a product of a crosslinking reaction. A cross-link is a bond or a short sequence of bonds that links one polypeptide chain to another. Crosslinking may occur naturally or synthetically. Examples of cross-links include, for example, disulfide bonds, amine bonds, ester bonds, or imine bonds. A crosslinking reaction may increase or decrease the total molecular weight of crosslinked molecules relative to the unmodified molecules, depending on the crosslinking mechanism. In an exemplary embodiment, a size variant of a protein of interest, for example a HMW species of an ADC, is a dimer formed by a cross-link. In some exemplary embodiments, a cross-link is formed between a glutamine residue and a lysine residue. In some particular embodiments, a cross-linking lysine is located at the C-terminus of a protein of interest. In some exemplary embodiments, a cross-link is formed through an enzymatic reaction, for example by the action of microbial transglutaminase. In some exemplary embodiments, cross-linking may cause the formation of a dimer species, a trimer species, a tetramer species, or larger species of a protein of interest, for example an ADC.

As used herein, the term “crosslinking agent” refers to a molecule capable of forming a cross-link between two or more other molecules, or between two or more sites on one or more molecules. In some exemplary embodiments, a crosslinking agent is an enzyme (“crosslinking enzyme”), for example mTG.

The chromatographic material for SEC can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

Porous chromatographic resins appropriate for size-exclusion chromatography of viruses may be made of dextrose, agarose, polyacrylamide, or silica which have different physical characteristics. Polymer combinations can also be also used. Most commonly used are those under the tradename “SEPHADEX” available from Amersham Biosciences. Other size exclusion supports from different materials of construction are also appropriate, for example Toyopearl 55F (polymethacrylate, from Tosoh Bioscience, Montgomery Pa.) and Bio-Gel P-30 Fine (BioRad Laboratories, Hercules, Calif.).

In some exemplary embodiments, the mobile phase used to obtain said eluate from size exclusion chromatography can comprise a volatile salt. In some specific embodiments, the mobile phase can comprise ammonium acetate, ammonium bicarbonate, or ammonium formate, or combinations thereof.

As used herein, the term “mass spectrometer” includes a device capable of identifying specific molecular species and measuring their accurate masses. The term is meant to include any molecular detector with which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on, as long as one can obtain meaningful information, or the fragment ion signal is detectable. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device. The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

As used herein, the term “mass analyzer” includes a device that can separate species, that is, atoms, molecules, or clusters, according to their mass. Non-limiting examples of mass analyzers that could be employed are time-of-flight (TOF), magnetic electric sector, quadrupole mass filter (Q), quadrupole ion trap (QIT), orbitrap, Fourier transform ion cyclotron resonance (FTICR), and also the technique of accelerator mass spectrometry (AMS).

In some exemplary aspects, the mass spectrometer can work on nanoclectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoclectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary embodiments, the mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Acbersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion is selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

In some exemplary embodiments, LC-MS can be performed under native conditions. As used herein, the term “native conditions” can include performing mass spectrometry under conditions that preserve non-covalent interactions in an analyte. Native mass spectrometry is an approach to study intact biomolecular structure in the native or near-native state. The term “native” refers to the biological status of the analyte in solution prior to subjecting to the ionization. Several parameters, such as pH and ionic strength, of the solution containing the biological analytes can be controlled to maintain the native folded state of the biological analytes in solution. Commonly, native mass spectrometry is based on electrospray ionization, wherein the biological analytes are sprayed from a nondenaturing solvent. Other terms, such as noncovalent, native spray, electrospray ionization, nondenaturing, macromolecular, or supramolecular mass spectrometry can also be describing native mass spectrometry. In exemplary embodiments, native MS allows for better spatial resolution compared to non-native MS, improving detection of biotransformation products of a therapeutic protein. For detailed review on native MS, refer to the review: Elisabetta Boeri Erba & Carlo Pe-tosa, The emerging role of native mass spectrometry in characterizing the structure and dynamics of macromolecular complexes, 24 PROTEIN SCIENCE1176-1192 (2015).

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsf.edu/prospector/mshome.htm), Byonic (www.proteinmetrics.com/products/byonic) or Sequest (fields.scripps.edu/sequest).

It is understood that the present invention is not limited to any of the aforesaid protein(s), antibody(s), monoclonal antibody(s), bispecific antibody(s), protein expression system(s), antibody fragment(s), conjugated peptide(s) or protein(s), antibody-drug conjugate(s), conjugation site(s), cross-link(s), high molecular weight species, protein alkylating agent(s), protein denaturing agent(s), protein reducing agent(s), digestive enzyme(s), hydrolyzing agent(s), sample(s), liquid chromatography system(s), mobile phase(s), mass spectrometer(s), database(s), or bioinformatics tool(s), and any protein(s), antibody(s), monoclonal antibody(s), bispecific antibody(s), protein expression system(s), antibody fragment(s), conjugated peptide(s) or protein(s), antibody-drug conjugate(s), conjugation site(s), cross-link(s), high molecular weight species, protein alkylating agent(s), protein denaturing agent(s), protein reducing agent(s), digestive enzyme(s), hydrolyzing agent(s), sample(s), liquid chromatography system(s), mobile phase(s), mass spectrometer(s), database(s), or bioinformatics tool(s) can be selected by any suitable means.

The present invention will be more fully understood by reference to the following Examples. They should not, however, be construed as limiting the scope of the invention.

### Examples

Chemicals and reagents. All mAbs and ADCs were produced at Regeneron (Tarrytown, NY, USA). Acetonitrile (ACN; LC-MS grade), trifluoroacetic acid, formic acid, dithiothreitol (DTT), iodoacetamide (IAA), Tris-(2-carboxyethyl) phosphine hydrochloride (TCEP), 8M guanidine-HCl solution, and Invitrogen UltraPure 1 M Tris-HCl buffer, pH 7.5 were purchased from Thermo Fisher Scientific (Waltham, MA, USA).

Microbial transglutaminase (mTG) was purchased from MilliporeSigma (Burlington, MA, USA). Urea and carboxypeptidase B (CpB) were purchased from Sigma-Aldrich (St. Louis, MO, USA). Sequencing grade-modified trypsin was purchased from Promega (Madison, WI, USA). C18 (ACQUITY Ultra-performance LC peptide BEH C18 1.7 μm, 2.1 mm×150 mm) and phenyl columns (ACQUITY Ultra-performance LC BEH Phenyl 1.7 μm, 2.1 mm×150 mm) were purchased from Waters (Milford, MA, USA). FabRICATOR was purchased from Genovis (Lund, Sweden). PNGase F was purchased from New England Biolabs (Ipswich, MA, USA). Deionized water was provided by a Milli-Q integral water purification system installed with a MilliPak Express 20 filter (MilliporeSigma, Burlington, MA, USA).

Preparation of mAb and ADC samples. To evaluate crosslinking in mAb samples, mAb1 and mAb2 were incubated with 1 U/mg Ab of mTG in conjugation buffer at 37° C. for 1, 3, or 6 hours. To generate ADCs, mAb1 and mAb2 were incubated with mTG and linker payload (LP) in conjugation buffer at 37° C. for 6 hours. Control samples were incubated in conjugation buffer for 37° C. for 6 hours. The HC C-terminal K in mAb2 was enzymatically removed by digestion with carboxypeptidase B (CpB) at 37° C. for 2 hours. The CpB enzyme and baseline aggregates were then removed by SEC purification; this was followed by incubation with mTG alone or both mTG and the LP in conjugation buffer at 37° C. for 6 hours. All of the reaction mixtures resulting from the above were subjected to SEC analysis immediately thereafter or frozen at −80° C. for LC-MS peptide mapping or subunit analysis at a later date.

SEC analysis. Five μg of mAb or ADC samples were loaded onto an SEC column which separated the reaction products by size. Peaks eluted from the column were detected by UV absorption at 280 nm. Empower software (Waters) was used to perform peak integration to determine peak area and to calculate relative abundance (percent area).

LC-MS/MS peptide mapping analysis. mAb and ADC samples were denatured, then reduced, and alkylated with TCEP and IAA, respectively. The reduced samples were digested with trypsin for 4 hours at 37° C. followed by quenching with TFA. Tryptic digests (5 μg samples) were loaded onto a C18 column.

Peptides eluted from the C18 column were analyzed by UV absorption at 214 nm and subjected to MS acquisition using a Q Exactive™ Plus Hybrid Quadrupole-Orbitrap™ Mass Spectrometer (Thermo). The source parameters were set as follows: spray voltage, 3.8 kV; auxiliary gas, 10; auxiliary gas temperature, 250° C.; capillary temperature, 320° C.; and S-lens RF level, 50. Data-dependent acquisition (DDA) was performed with one full MS scan from 300 m/z to 2000 m/z followed by five sequential MS/MS scans for the five ions. Full MS scans were collected at a resolution of 70,000 with AGC target of 1E6. The MS/MS scans were collected at a resolution of 17,500 with an AGC target of 1E5. The isolation width was 4 m/z and the normalized collision energy (NCE) was set at 27.

LC-MS subunit analysis. mAb and ADC samples were digested with FabRICATOR® and deglycosylated with PNGase F at 37° C. for 2 hours. After digestion, the samples were denatured and reduced with guanidine HCl and DTT, respectively, and incubated at 60° C. for 30 minutes. Twenty μg samples of each preparation were injected onto a phenyl column (reversed phase column).

Subunits eluting from the phenyl column were detected by UV absorption at 280 nm and then subjected to MS acquisition on an X500B Quadrupole Time-Of-Flight (TOF) Mass Spectrometer (AB Sciex, Framingham, MA, USA). Full MS scans were collected from 500 m/z to 5000 m/z with the intact protein mode turned on. The ion source, curtain, and CAS gases were set at 60, 40, and 10 psi, respectively. The source temperature was set at 450° C. with a spray voltage of 5500 V and a declustering potential of 150 V.

Data Analysis. Peptides were identified by database searches against mAb sequences using Byonic (version 4.6.1, Protein Metrics, San Carlos, CA). Common mAb post-translational modifications (PTMs) were included in the search parameters as variable modifications; carbamidomethylation of cysteine was included as a fixed modification. Q-K crosslinking associated with a loss of NH3 (−17.0265 Da), glutamine deamidation (+0.9840 Da), and conjugation of LP were included as variable modifications as well. Peptide quantification was performed using Skyline software (version 21.2, MacCoss Lab Software, Seattle, WA, USA). Analysis of subunit data was conducted using Sciex OS software (version 2.0 AB Sciex Framingham, MA, USA). The Bio Tool Kit was used for protein deconvolution.

### Example 1. Characterization of High Molecular Weight Species of Antibody-Drug Conjugates

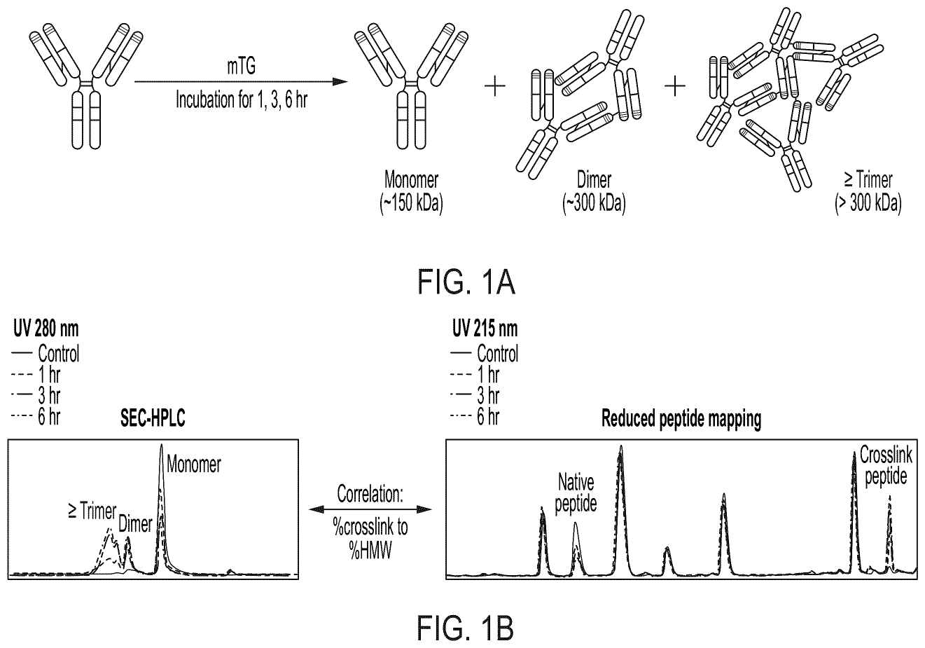

To study the relationship between the formation of HMW variants and mTG-mediated reactions, two mAbs (mAb1 and mAb2), each containing target glutamines engineered for site-specific conjugation, were selected as model systems. While mTG-mediated conjugation of mAb1 typically results in a high level of HMW variants, conjugation of mAb2 yielded a comparatively low level of HMW variants. As shown in FIG. 1A, both mAbs were incubated with mTG alone in the absence of LP to maximize the level of HMW variants to facilitate the identification of cross-links. After incubation of mAb1 with mTG alone for 1, 3, and 6 hours, the percentage of HMW variants determined by SEC increased from 6.1% in the control sample to 46.6%, 69.3%, and 74.3%, respectively, as shown in the left panel of FIG. 1B. These high levels of HMW variants enable confident identification of cross-links by peptide mapping without the need for enrichment or specialized workflows. As shown in the right panel of FIG. 1B, the change in the abundance of the native peptide and the most prominent of the crosslinked peptides can be readily observed in UV profiles generated by reduced peptide mapping. Finally, the increased percentage of HMW variants detected by SEC over time was compared with the percentage of crosslinked peptides quantified from peptide mapping studies to establish a correlation between the formation of HMW variants and mTG-mediated crosslinking.

### Example 2. Identification of Off-Target mTG Crosslinking

Reaction mixtures generated by incubation of mAb1 and mAb2 with mTG were analyzed by reduced peptide mapping to identify crosslinked peptides. The results of database searching and manual data interpretation confirmed that both mAbs underwent mTG-mediated Q-K crosslinking. The crosslinked sites were successfully identified in samples generated from both mAb1 and mAb2.

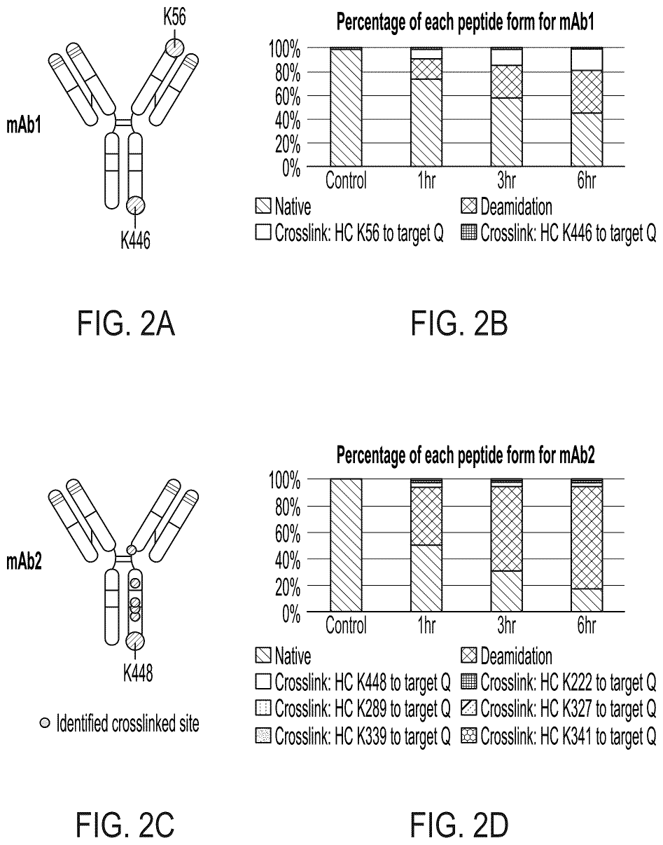

As described in Example 1, analysis using SEC revealed that mTG incubation of mAb1 resulted in a significant increase in the level of HMW variants. As shown in FIG. 2A, most of the crosslinks detected in mAb1 were between HC K56 or K446 (the HC C-terminal K) and the engineered target glutamine. After incubation with mTG, the mAb1 target Q was found in one of three distinct forms: native, deamidated, or crosslinked. These observations are consistent with the current understanding of mTG-mediated reactions. The abundance of each form that included the target Q was determined based on their peak area and corresponding accurate peptide masses, as shown in Table 1. As shown in FIG. 2B, HC K56 was detected in a higher fraction of the crosslinked peptides than HC K446. The total percentage of crosslinked peptides detected at the target Q increased significantly with longer incubation times; this increase was due primarily to increases in crosslinking between target Q and HC K56 over time. In addition to crosslinking, it was also observed that deamidation of the target Q increased over time. This was as expected, given that mTG catalyzes Q deamidation in the absence of primary amine-containing substrates, such as a primary amine-containing LP.

TABLE 1
Relative abundance of each mAb1 peptide that
included the target glutamine (Q), including
native, deamidated, and crosslinked forms.

| Peptide form | Control | 1 hr | 3 hr | 6 hr |
|---|---|---|---|---|
| Native | 99.8% | 73.0% | 57.5% | 45.3% |
| Deamidated | 0.2% | 17.4% | 27.8% | 35.6% |
| Crosslink: HC K56 to target Q | ND | 8.3% | 13.6% | 18.2% |
| Crosslink: HC K446 to target Q | ND | 1.3% | 1.0% | 0.9% |
| Total Crosslinked | ND | 9.6% | 14.6% | 19.0% |

ND: Not detected.

Based on these findings, the highly reactive HC K56 residue can be eliminated from the mAb1 backbone, for example by mutation of the encoding nucleic acid sequence. This small change might result in a significant reduction in the level of HMW variants that are generated and may lead to an improvement in the overall quality of the ADC product. However, a mutation introduced at HC K56 might have an undesirable impact on other physiochemical properties, and would thus need to undergo a thorough evaluation.

The mAb2 amino acid sequence does not include HC K56 and generates a much lower increase in the level of HMW variants during mTG-mediated conjugation. In contrast to what was observed for mAb1, the mAb2 HC K448 (C-terminal K) was identified as the predominant crosslinked site, as shown in FIG. 2C. Other minor crosslinked sites identified in the Fc and Fd regions of the HC were detected at a substantially lower abundance, as shown in Table 2. Unlike mAb1, the abundance of total crosslinks detected in mAb2 that included the target Q changed minimally over time, as shown in FIG. 2D. On the other hand, the increase in mTG-mediated deamidation of target Q was much more significant in mAb2 compared to mAb1. These results suggest that, in the absence of a highly reactive lysine residue (for example, HC K56 in mAb1), deamidation is more kinetically favorable than Q-K crosslinking.

TABLE 2
Relative abundance of each mAb2 peptide that
included the target glutamine (Q), including
native, deamidated, and crosslinked forms.

| Peptide form | Control | 1 hr | 3 hr | 6 hr |
|---|---|---|---|---|
| Native | 100.0% | 49.8% | 31.1% | 17.4% |
| Deamidated | 0.0% | 44.8% | 63.9% | 77.7% |
| Crosslink: HC K448 to target Q | ND | 4.6% | 4.2% | 4.0% |
| Crosslink: HC K222 to target Q | ND | 0.6% | 0.5% | 0.5% |
| Crosslink: HC K289 to target Q | ND | 0.1% | 0.1% | 0.2% |
| Crosslink: HC K327 to target Q | ND | 0.0% | 0.0% | 0.0% |
| Crosslink: HC K339 to target Q | ND | 0.1% | 0.1% | 0.1% |
| Crosslink: HC K341 to target Q | ND | 0.0% | 0.0% | 0.0% |
| Total Crosslinked | ND | 5.5% | 5.1% | 4.9% |

ND: Not detected.

The lack of multiple reactive lysine residues in these mAbs was not unexpected. It has been previously reported that without specific lysine substitution, only the HC C-terminal K can serve as an efficient site for conjugation with glutamine-based ligands (Spidel, J. L., and Albone, E. F. (2019) Efficient Production of Homogeneous Lysine-Based Antibody Conjugates Using Microbial Transglutaminase, Methods in molecular biology (Clifton, N.J.) 2033, 53-65; Spidel, J. L., et al. (2017) Site-Specific Conjugation to Native and Engineered Lysines in Human Immunoglobulins by Microbial Transglutaminase, Bioconjugate chemistry 28, 2471-2484). In this Example, an additional lysine (HC K56) was identified in mAb1 that can form crosslinks with the target Q. These results suggest that, although this phenomenon may not be commonly observed, certain native lysine residues in some mAbs may be effectively conjugated with glutamine-based ligands, and the method of the present invention can be used to identify these reactive lysines.

### Example 3. Correlation Between the Level of HMW Variants and the Extent of mTG-Mediated Crosslinking

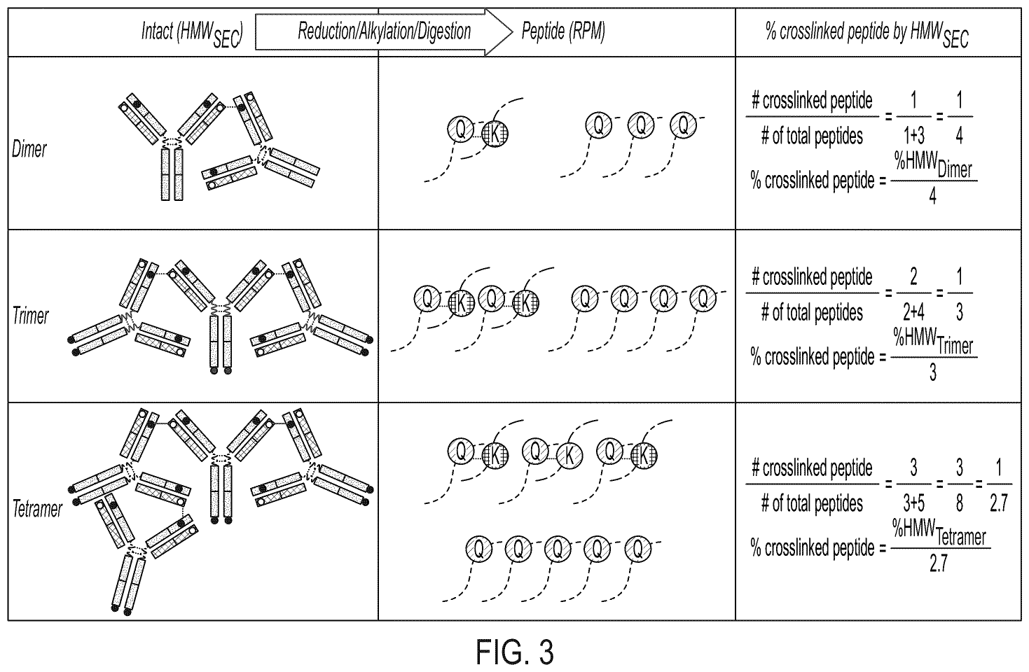

The relationship between the percentage of crosslinked peptides determined by peptide mapping (% crosslink by RPM) and the percentage of HMW variants determined by SEC (% HMWSEC) was evaluated to determine whether mTG-mediated crosslinking was a primary contributor to the elevated level of HMW variants observed in mTG-mediated conjugation. A correction factor was introduced to convert the percentage of HMW variants determined by SEC, which was measured on the intact level, to percentage of crosslinked peptides, so that the percentage of HMW variants determined by SEC can be correlated with the percentage of crosslinked peptides determined by RPM, which was measured on the peptide level. The model for converting % HMWSEC to % crosslink is based on the observation that there was no % HMW increase in the control sample (as shown in FIG. 1B), and therefore it can be assumed that all HMW species are caused by Q-K crosslinking. As shown in FIG. 3, one of four, two of six, and three of eight target Q-containing peptides were crosslinked in dimers, trimers, and tetramers, respectively.

Based on this observation, the % HMWdimer can be converted into % crosslinked peptide by dividing the former value by a correction factor of four; similarly, % HMWtrimer can be converted into % crosslinked peptide by dividing by three. Similar conversions can be performed for all HMW species; correction factors for each HMW species up to 10-mer are listed in Table 3. As a result, in order to convert the percentage of HMW variants determined by SEC to percentage of crosslinked peptides, the dimer peak identified by SEC was adjusted using the aforementioned correction factor of 4, while the peaks for all other HMW species were adjusted using the correction factor of 2.7, because the value of this correction factor changed minimally from tetramer to 10-mer states (see Equation 1).

TABLE 3
Correction factors for calculating the percentage of crosslinked peptide
from the percentage of HMW variants for different HMW forms.

| HMW form | Correction factor |
|---|---|
| Dimer | 4 |
| Trimer | 3 |
| Tetramer | 2.7 |
| Pentamer | 2.5 |
| Hexamer | 2.4 |
| 10-mer | 2.2 |

Equation 1: Calculation used to estimate % crosslinked peptides from % HMW species. All values shown were determined by SEC.

$$\% crosslink by {HMW}_{SEC} \approx$$

$$[ \% {HMW}_{Dimer} / 4 ] + [ ( \% {HMW}_{Total} - \% {HMW}_{Dimer} ) / 2.7 ]$$

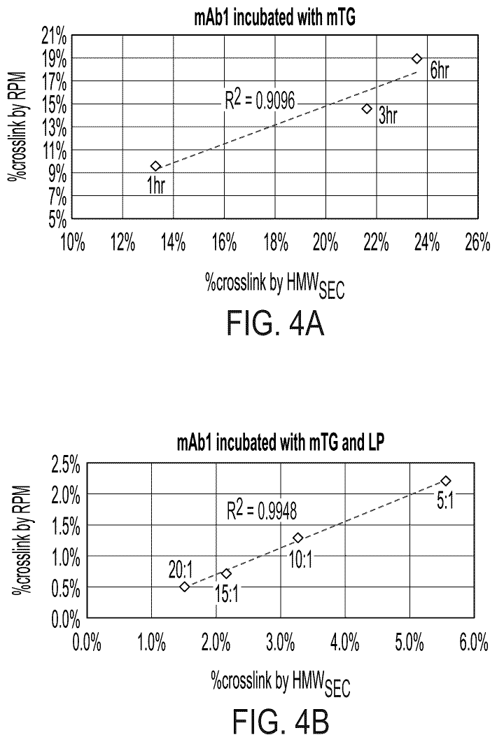

The percentage of crosslinked peptides measured by peptide mapping and values converted from SEC using Equation 1 are both included in Table 4 and Table 5. The results shown in FIG. 4A demonstrate a strong correlation between the measured and predicted percentage of crosslinked peptides in mAb1 after incubation with mTG for 1, 3, and 6 hours. These results suggest that the increase in the level of HMW variants of mAb1 observed after mTG-mediated conjugation can be attributed primarily to the formation of Q-K crosslinks.

TABLE 4
HMW species and crosslinked peptides
for mAb1 incubated with mTG

| | | 1 hr | 3 hrs | 6 hrs |
|---|---|---|---|---|
| SEC | HMW_{Total} | 46.6% | 69.3% | 74.3% |
| | HMW_{Dimer} | 19.3% | 20.0% | 19.0% |
| | % crosslinked peptide by HMW_{SEC} | 13.3% | 21.6% | 23.6% |
| RPM | % crosslinked peptide by RPM | 9.6% | 14.6% | 19.0% |

TABLE 5
HMW species and crosslinked peptides for mAb1 incubated
with mTG and LP, by molar ratio of LP:mAb

| | | 5:1 | 10:1 | 15:1 | 20:1 |
|---|---|---|---|---|---|
| SEC | HMW_{Total} | 25.0% | 17.4% | 13.6% | 11.3% |
| | HMW_{Dimer} | 17.2% | 12.9% | 10.3% | 8.6% |
| | % crosslinked peptide by | 5.6% | 3.3% | 2.2% | 1.5% |
| | HMW_{SEC} | | | | |
| RPM | % crosslinked peptide by | 2.2% | 1.3% | 0.7% | 0.5% |
| | RPM | | | | |

Additional experiments were performed in which mAb1 was incubated with both mTG and LP. All crosslinked peptides identified in the incubations that included mTG alone were also detected in the incubations that included both mTG and the LP (producing ADCs), albeit at much lower abundances. There was also a good correlation between values for the percentage of crosslinked peptides for these ADC samples quantified using SEC and peptide mapping methods, as shown in FIG. 4B. These results also support the conclusion that the HMW variants, whether in mAbs or ADCs, result primarily from mTG-mediated Q-K crosslinking.

### Example 4. Removal of the HC C-Terminal Lysine Reduces the Level of HMW Variants Detected in mTG-Mediated ADCs

Although a good correlation was observed between the level of HMW variants and the extent of mTG-mediated Q-K crosslinking in mAb1, a similarly strong correlation was not identified in the case of mAb2. While mAb1 has two reactive K residues, the HC C-terminal K is the only major crosslinking site in mAb2. Results from a previous study revealed that removal of the HC C-terminal K can completely abolish crosslinking between the two HCs that occurs during mTG-mediated conjugation (Siegmund, V., et al. (2015) Locked by Design: A Conformationally Constrained Transglutaminase Tag Enables Efficient Site-Specific Conjugation, Angewandte Chemie (International ed. in English) 54, 13420-13424). However, the relationship between the HC C-terminal K and the generation of crosslinked peptides and HMW variants has not been investigated extensively.

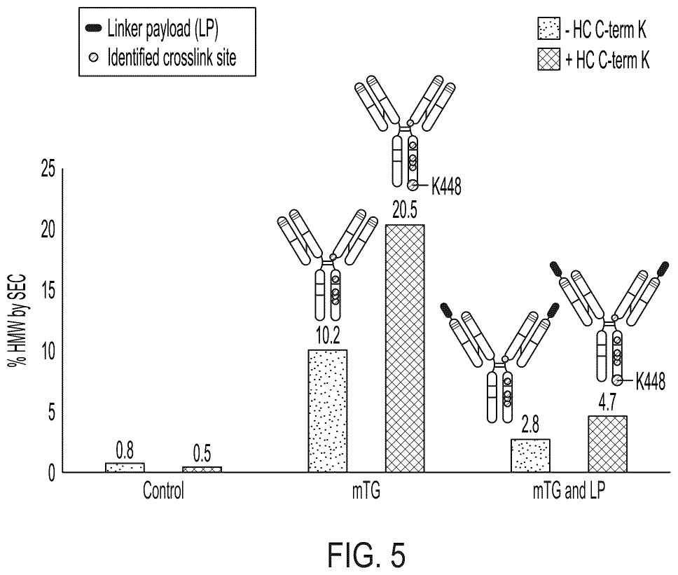

In order to determine how the HC C-terminal K contributed to the formation of HMW species during mTG-mediated crosslinking, carboxypeptidase B (CpB) was used to remove the HC C-terminal K from mAb2. The resulting changes in the percentage of HMW variants formed during mTG-mediated conjugation were compared. As shown in FIG. 5, after incubation with mTG alone, the percentage of HMW variants detected in mAb2 with no HC C-terminal K decreased by 50% compared to mAb2 with intact HC C-terminal K residues. This decrease was 40% in response to incubation with both mTG and the LP. These results indicate that the removal of HC C-terminal K can significantly reduce the level of HMW variants in the resulting ADCs, and suggest that the HC C-terminal K is the major contributor to mTG-mediated crosslinking in mAb2. Although only 10% of mAb2 contains HC C-terminal K before CpB treatment and these residues are responsible for only about a ˜2% increase in the percentage of HMW variants detected, this can change from process to process. Thus, HC C-terminal K represents a potential risk for the formation of HMW variants, and its removal should be considered in future drug development strategies.

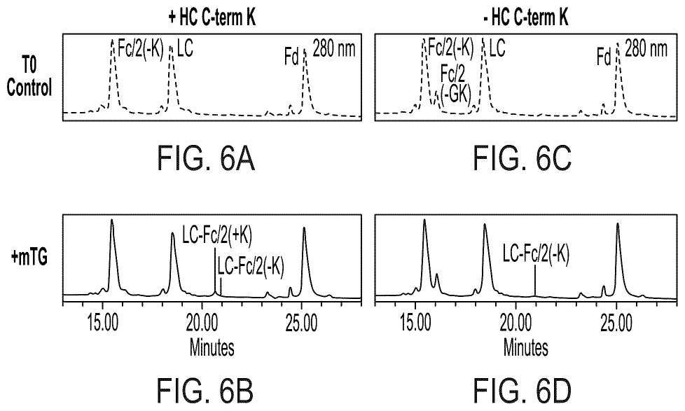

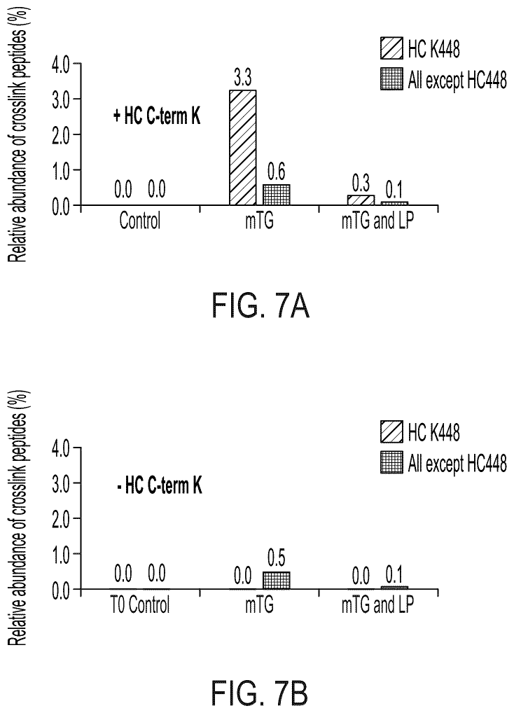

The incubation samples described above were subjected to extended characterization, including both subunit analysis and peptide mapping. Subunit analysis revealed that LC-Fc/2 (+HC C-terminal K) was the major crosslinked fragment in the untreated mAb, as shown in FIG. 6B. This observation was consistent with peptide mapping results shown in FIG. 7, in which the HC C-terminal K was identified as the only major site contributing to crosslinking. Comparatively low levels of LC-Fc/2 (−HC C-terminal K) were observed in both CpB-treated and untreated samples with signals of similar intensity, as shown in FIG. 6B and FIG. 6D. These results indicate that the removal of HC C-terminal K does not render any of the other lysine residues more susceptible to mTG-mediated crosslinking. The peptide mapping results also revealed low-level crosslinking at minor sites on HC. This result suggests that K-crosslinked sites may exist across the mAb backbone and may explain why an increase in the percentage of HMW variants was observed even after the complete removal of the HC C-terminal K.

This disclosure sets forth methods for determining the mechanisms underlying the formation of HMW variants in mTG-mediated ADCs using SEC and LC-MS techniques. Using the methods of the present invention, it was found that mTG-mediated Q-K crosslinking is the primary source of an increased level of HMW variants in ADCs, thus affecting the quality of the final ADC products. Q-K crosslinking sites were identified in the two mAbs studied. A correlation was established between the percentage of HMW variants determined by SEC and the percentage of crosslinked peptides quantified by peptide mapping. More importantly, it was demonstrated that the level of HMW variants was substantially reduced in the ADC conjugated from CpB-treated mAb2, in which the HC C-terminal K was complete removed. This result directly demonstrates that the HC C-terminal K was the major contributor to crosslinking and the formation of HMW variants in mAb2.

Crosslinking and other side reactions that occur during mTG-mediated conjugation need to be carefully monitored and controlled to maintain the high quality and consistency of ADC products. The identification of reactive lysine residues, including HC K56 in mAb1 and HC C-terminal K in both mAbs, provides insight into how mAbs may be designed to minimize the formation of HMW variants in the resulting ADCs. For example, highly reactive sites such as HC K56 in mAb1 should be eliminated during the early developmental stages by screening the ADC candidates based on their level of HMW variants. Extended characterization using LC-MS techniques, such as peptide mapping, can be performed if localization and mutation of these residues prove to be necessary. Removal of the HC C-terminal K may be a preferred route to minimize the formation of HMW variants and to avoid future complications in ongoing product development.

What is claimed is:

1. A method for producing an antibody-drug conjugate with reduced high molecular weight (HMW) species, the method comprising:
(a) contacting an antibody including a C-terminal lysine to a carboxypeptidase to produce a clipped antibody, wherein said clipped antibody does not include said C-terminal lysine; and
(b) contacting said clipped antibody to a crosslinking agent and a linker-payload to produce an antibody-drug conjugate with reduced HMW species.

2. The method of claim 1, wherein said carboxypeptidase is a metallo-carboxypeptidase, serine carboxypeptidase, cysteine carboxypeptidase, carboxypeptidase A, carboxypeptidase B, carboxypeptidase C, carboxypeptidase D, or carboxypeptidase E.

3. The method of claim 1, wherein said carboxypeptidase is carboxypeptidase B.

4. The method of claim 1, wherein contacting said antibody to said carboxypeptidase is conducted for about 1 hour to about 3 hours at about 35° C. to about 39° C.

5. The method of claim 1, wherein said payload is a cytotoxic payload or a therapeutic payload.

6. The method of claim 1, wherein said antibody includes a glutamine engineered for site-specific conjugation, optionally wherein said crosslinking agent is capable of crosslinking said glutamine and said C-terminal lysine.

7. The method of claim 1, wherein said crosslinking agent is an enzyme, optionally wherein said enzyme is microbial transglutaminase.

8. A method for producing an antibody-drug conjugate with reduced high molecular weight (HMW) species, the method comprising:
(a) identifying at least one off-target amino acid residue that forms crosslinks in an antibody-drug conjugate, said identifying comprising:
(i) contacting a sample including an antibody to a linker and a crosslinking agent to produce a crosslinked sample, wherein said crosslinking agent is capable of crosslinking said antibody at a target amino acid residue to said linker, and wherein said crosslinking agent is capable of crosslinking said antibody at a target amino acid residue to said antibody at an off-target amino acid residue;
(ii) contacting said crosslinked sample to at least one digestive enzyme to produce a peptide digest;
(iii) subjecting said peptide digest to liquid chromatography-mass spectrometry (LC-MS) analysis to identify crosslinked peptides;
(iv) using said identification to identify at least one off-target amino acid residue that forms crosslinks;
(b) contacting said antibody to at least one protease to produce a clipped antibody, wherein said clipped antibody does not include said at least one identified off-target amino acid residue; and
(c) contacting said clipped antibody to a linker and said crosslinking agent to produce an antibody-drug conjugate with reduced high molecular weight species.

9. The method of claim 8, wherein said at least one off-target amino acid residue is a lysine, optionally wherein said lysine is a C-terminal lysine.

10. The method of claim 8, wherein said target amino acid residue is a lysine, a cysteine, an unnatural amino acid, or a glutamine.

11. The method of claim 8, wherein said target amino acid residue is engineered for site-specific conjugation.

12. The method of claim 8, wherein said linker is attached to a payload, optionally wherein said payload is a cytotoxic payload or a therapeutic payload.

13. The method of claim 8, wherein said crosslinking agent is an enzyme, optionally wherein said crosslinking agent is microbial transglutaminase (mTG).

14. The method of claim 8, wherein said at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C), outer membrane protein T (OmpT), immunoglobulin-degrading enzyme of Streptococcus pyogenes (IdeS), thermolysin, papain, pronase, V8 protease, variants thereof, biologically active fragments thereof, homologs thereof, or combinations thereof.

15. The method of claim 8, wherein said at least one digestive enzyme is trypsin.

16. The method of claim 8, wherein said at least one digestive enzyme is IdeS or a variant thereof.

17. The method of claim 8, wherein said liquid chromatography is selected from a group consisting of reverse phase liquid chromatography, anion exchange chromatography, cation exchange chromatography, size exclusion chromatography, affinity chromatography, hydrophobic interaction chromatography, hydrophilic interaction chromatography, and mixed-mode chromatography.

18. The method of claim 8, wherein said LC-MS analysis is RPLC-MS/MS analysis.

19. The method of claim 8, wherein said at least one protease is a carboxypeptidase, optionally wherein said carboxypeptidase is carboxypeptidase B.

20. The method of claim 8, wherein contacting said antibody to said at least one protease is conducted for about 1 hour to about 3 hours at about 35° C. to about 39° C.

21. The method of claim 8, wherein said clipped antibody is an antibody lacking a C-terminal lysine.

22. A method for characterizing crosslinking sites in a protein of interest, the method comprising:
(a) contacting a sample including a protein of interest to a crosslinking agent to produce a crosslinked protein of interest, wherein said protein of interest includes at least one target amino acid residue that can be crosslinked by said crosslinking agent;
(b) contacting said crosslinked protein of interest to at least one digestive enzyme to produce a peptide digest;
(c) subjecting said peptide digest to liquid chromatography-mass spectrometry (LC-MS) analysis to characterize peptides that include a crosslink at said at least one target amino acid residue; and
(d) using said characterized peptides to characterize crosslinking sites in said protein of interest.

23. The method of claim 22, wherein said protein of interest is an antibody, a bispecific antibody, an antibody fragment, an antibody-drug conjugate, a fusion protein, or a recombinant protein.

24. The method of claim 22, wherein said crosslinking agent is an enzyme, optionally wherein said enzyme is microbial transglutaminase (mTG).

25. The method of claim 22, wherein said target amino acid residue is a lysine, a cysteine, an unnatural amino acid, or a glutamine.

26. The method of claim 22, wherein said target amino acid residue is engineered for site-specific conjugation.

27. The method of claim 22, wherein step (a) further comprises contacting said protein of interest and said crosslinking agent to a linker, wherein said crosslinking agent is capable of crosslinking said protein of interest to said linker.

28. The method of claim 27, wherein said linker is attached to a payload, optionally wherein said payload is a cytotoxic payload or a therapeutic payload.

29. The method of claim 22, wherein said at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C), outer membrane protein T (OmpT), immunoglobulin-degrading enzyme of Streptococcus pyogenes (IdeS), thermolysin, papain, pronase, V8 protease, variants thereof, biologically active fragments thereof, homologs thereof, or combinations thereof.

30. The method of claim 22, wherein said at least one digestive enzyme is trypsin.

31. The method of claim 22, wherein said at least one digestive enzyme is IdeS or a variant thereof.

32. The method of claim 22, wherein said liquid chromatography is selected from a group consisting of reverse phase liquid chromatography, anion exchange chromatography, cation exchange chromatography, size exclusion chromatography, affinity chromatography, hydrophobic interaction chromatography, hydrophilic interaction chromatography, and mixed-mode chromatography.

33. The method of claim 22, wherein said LC-MS analysis is RPLC-MS/MS analysis.

34. The method of claim 22, wherein said crosslinking sites include a lysine, optionally wherein said lysine is a C-terminal lysine.

35. The method of claim 22, wherein characterizing said crosslinking sites includes identifying amino acid residues that crosslink to said target amino acid residue.

36. A method for identifying at least one reactive lysine in a protein of interest, the method comprising:
(a) contacting a sample including a protein of interest to a crosslinking agent to produce a crosslinked protein of interest, wherein said crosslinking agent is capable of crosslinking at least one amino acid residue in said protein of interest to at least one reactive lysine in said protein of interest;
(b) contacting said crosslinked protein of interest to at least one digestive enzyme to produce a peptide digest;
(c) subjecting said peptide digest to liquid chromatography-mass spectrometry (LC-MS) analysis to identify peptides that include a crosslink between said at least one amino acid residue and at least one reactive lysine; and
(d) using said identified peptides to identify said at least one reactive lysine in said protein of interest.

37. The method of claim 36, wherein said protein of interest is an antibody, a bispecific antibody, an antibody fragment, an antibody-drug conjugate, a fusion protein, or a recombinant protein.

38. The method of claim 36, wherein said crosslinking agent is an enzyme, optionally wherein said enzyme is microbial transglutaminase (mTG).

39. The method of claim 36, wherein said reactive lysine is a C-terminal lysine.

40. The method of claim 36, wherein said at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C), outer membrane protein T (OmpT), immunoglobulin-degrading enzyme of Streptococcus pyogenes (IdeS), thermolysin, papain, pronase, V8 protease, variants thereof, biologically active fragments thereof, homologs thereof, or combinations thereof.

41. The method of claim 36, wherein said at least one digestive enzyme is trypsin.

42. The method of claim 36, wherein said at least one digestive enzyme is IdeS or a variant thereof.

43. The method of claim 36, wherein said liquid chromatography is selected from a group consisting of reverse phase liquid chromatography, anion exchange chromatography, cation exchange chromatography, size exclusion chromatography, affinity chromatography, hydrophobic interaction chromatography, hydrophilic interaction chromatography, and mixed-mode chromatography.

44. The method of claim 36, wherein said LC-MS analysis is RPLC-MS/MS analysis.

45. A method for determining a contribution of site-specific crosslinking to high molecular weight species of a protein of interest, the method comprising:
(a) subjecting a protein of interest to conditions suitable for promoting site-specific crosslinking to produce a crosslinked protein of interest;
(b) subjecting said crosslinked protein of interest to size exclusion chromatography (SEC) analysis to quantify a percent of high molecular weight (HMW) species;
(c) using said quantification to determine a predicted percent of site-specific crosslinked peptides that may contribute to said HMW species, using Equation 1;
(d) subjecting said crosslinked protein of interest of step (a) to peptide mapping analysis to quantify a percent of site-specific crosslinked peptides; and
(e) comparing said quantified percent of site-specific crosslinked peptides of step (d) to said predicted percent of site-specific crosslinked peptides of step (c) to determine a contribution of site-specific crosslinking to HMW species of said protein of interest.

46. The method of claim 45, wherein said protein of interest is an antibody, a bispecific antibody, an antibody fragment, an antibody-drug conjugate, a fusion protein, or a recombinant protein.

47. The method of claim 45, wherein said site-specific crosslinking comprises crosslinking of an engineered amino acid residue.

48. The method of claim 45, wherein subjecting a protein of interest to conditions suitable for promoting site-specific crosslinking includes contacting said protein of interest to a crosslinking agent.

49. The method of claim 47, wherein said crosslinking agent is an enzyme, optionally wherein said enzyme is microbial transglutaminase (mTG).

50. The method of claim 45, wherein said peptide mapping analysis includes contacting said crosslinked protein of interest to at least one digestive enzyme to produce a peptide digest, and then subjecting said peptide digest to RPLC-MS/MS analysis.

51. The method of claim 50, wherein said at least one digestive enzyme is trypsin.

52. A method for determining a contribution of a C-terminal lysine to formation of high molecular weight (HMW) species in an antibody-drug conjugate of interest, the method comprising:
(a) contacting an antibody corresponding to an antibody-drug conjugate of interest to a carboxypeptidase to produce a clipped antibody, wherein said antibody includes a C-terminal lysine and said clipped antibody does not include a C-terminal lysine;
(b) contacting said antibody and said clipped antibody to a crosslinking agent to produce a crosslinked antibody and a crosslinked clipped antibody, wherein said crosslinking agent is capable of crosslinking at least one amino acid residue of said antibody and of said clipped antibody to a lysine;
(c) subjecting said crosslinked antibody and said crosslinked clipped antibody to size exclusion chromatography (SEC) analysis to quantify HMW species of said crosslinked antibody and said crosslinked clipped antibody; and
(d) comparing said quantification of HMW species of said crosslinked antibody to said quantification of HMW species of said crosslinked clipped antibody to determine a contribution of a C-terminal lysine to formation of HMW species in said antibody-drug conjugate of interest.

53. The method of claim 52, wherein said carboxypeptidase is a metallo-carboxypeptidase, serine carboxypeptidase, cysteine carboxypeptidase, carboxypeptidase A, carboxypeptidase B, carboxypeptidase C, carboxypeptidase D, or carboxypeptidase E.

54. The method of claim 52, wherein said carboxypeptidase is carboxypeptidase B.

55. The method of claim 52, wherein said crosslinking agent is an enzyme, optionally wherein said crosslinking agent is microbial transglutaminase (mTG).

56. A method for selecting an antibody for an antibody-drug conjugate, the method comprising:
(a) obtaining a sample including a first antibody, wherein said first antibody comprises at least one target amino acid residue that may be crosslinked by a crosslinking agent to at least one off-target amino acid residue;
(b) contacting said first antibody to said crosslinking agent to produce a crosslinked antibody;
(c) contacting said crosslinked antibody to at least one digestive enzyme to produce a peptide digest;
(d) subjecting said peptide digest to liquid chromatography-mass spectrometry (LC-MS) analysis to quantify peptides comprising said at least one target amino acid residue crosslinked to at least one off-target amino acid residue for a first antibody;
(e) repeating steps (a)-(d) with at least one additional antibody to quantify peptides comprising at least one target amino acid residue crosslinked to at least one off-target amino acid residue for at least one additional antibody;
(f) comparing the quantifications of steps (d) and (e); and
(g) using said comparison to select an antibody for an antibody-drug conjugate.

57. The method of claim 56, wherein said at least one target amino acid residue is a lysine, a cysteine, an unnatural amino acid, or a glutamine.

58. The method of claim 56, wherein said at least one target amino acid residue is engineered for site-specific conjugation.

59. The method of claim 56, wherein said at least one off-target amino acid residue is a lysine, optionally wherein said lysine is a C-terminal lysine.

60. The method of claim 56, wherein said crosslinking agent is an enzyme, optionally wherein said crosslinking agent is microbial transglutaminase (mTG).

61. The method of claim 56, wherein step (b) further comprises contacting said first antibody and said crosslinking agent to a linker, wherein said crosslinking agent is capable of crosslinking said first antibody to said linker.

62. The method of claim 61, wherein said linker is attached to a payload, optionally wherein said payload is a cytotoxic payload or a therapeutic payload.

63. The method of claim 56, wherein said at least one digestive enzyme is selected from the group consisting of protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C), outer membrane protein T (OmpT), immunoglobulin-degrading enzyme of Streptococcus pyogenes (IdeS), thermolysin, papain, pronase, V8 protease, variants thereof, biologically active fragments thereof, homologs thereof, or combinations thereof.

64. The method of claim 56, wherein said at least one digestive enzyme is trypsin.

65. The method of claim 56, wherein said at least one digestive enzyme is IdeS or a variant thereof.

66. The method of claim 56, wherein said liquid chromatography is selected from a group consisting of reverse phase liquid chromatography, anion exchange chromatography, cation exchange chromatography, size exclusion chromatography, affinity chromatography, hydrophobic interaction chromatography, hydrophilic interaction chromatography, and mixed-mode chromatography.

67. The method of claim 56, wherein said LC-MS analysis is RPLC-MS/MS analysis.
